(19) United States
(12) Patent Application Publication
(10) Pub. No.: US 2024/0287495 A1
(43) Pub. Date: Aug. 29, 2024

(54) DEVICES AND METHODS FOR ISOLATING AND DETECTING VIRAL NUCLEIC ACIDS

(71) Applicant: University of Rochester, Rochester, NY (US)

(72) Inventors: Benjamin L. Miller, Rochester, NY (US); Jeffrey W. Beard, Rochester, NY (US)

(73) Assignee: University of Rochester, Rochester, NY (US)

(21) Appl. No.: 18/572,555

(22) PCT Filed: Jun. 23, 2022
(86) PCT No.: PCT/US22/34636

§ 371 Date: Dec. 20, 2023

Related U.S. Application Data
(60) Provisional application No. 63/215,002, filed on Jun. 25, 2021.

(51) Int. Cl. C12N 15/10; B01L 3/00; C12Q 1/6806

(52) U.S. Cl. CPC C12N 15/1003; B01L 3/502715; B01L 3/502761; C12Q 1/6806; B01L 2200/0621; B01L 2200/10; B01L 2300/0681; B01L 2300/0819; B01L 2300/087; B01L 2300/0874; B01L 2300/0877; B01L 2300/0887; B01L 2300/12

(57) ABSTRACT

The present invention relates to novel devices and methods for viral lysis and isolation as well as a simple, disposable, and nonenzymatic viral load assay based on a hairpin cascade reaction (HCR).

### CROSS-REFERENCE TO RELATED APPLICATIONS

This application is the U.S. national phase of International Patent Application No. PCT/US22/34636, filed Jun. 23, 2022, which claims priority under 35 U.S.C. § 119(e) to U.S. Provisional Patent Application No. 63/215,002, filed Jun. 25, 2021. The foregoing applications are incorporated by reference herein in their entirety.

### STATEMENT REGARDING FEDERALLY SPONSORED RESEARCH

This invention was made with government support under EB027049 by National Institutes of Health. The government has certain rights in the invention.

### REFERENCE TO A SEQUENCE LISTING

This application incorporates by reference the Sequence Listing submitted in Computer Readable Form as file SeqListing_161118-02901_ST25, created on Jun. 19, 2022 and containing 2,685 bytes.

### FIELD

This invention relates generally to devices and methods for isolating or detecting viral nucleic acids.

### BACKGROUND

Viruses cause various disorders and can spread rapidly throughout the globe. For example, there are approximately 37.9 million people living with the human immunodeficiency virus (HIV) worldwide and over a million new infections each year. As the number of people living with HIV increases, it becomes essential to develop economical and highly accessible clinical tests relevant to the disease. Viral load assays are used to measure the number of HIV virions in a patient's blood and are the most effective method to track an HIV patient's health and state of infection. Patients placed on antiretroviral therapy (ART) are recommended to have their viral load tested every 3 months for the United States or every 6-12 months to meet the WHO's global standard. Quantitative reverse transcription polymerase chain reaction (RT-qPCR) is the gold standard test for determining an HIV viral load. While developed countries such as the United States can afford frequent viral loading and CD4 count tests, the majority of the world's HIV-positive population has restricted access to the finances, supplies, and laboratories necessary to perform these assays. Low-to-middle income countries (LMICs) have the greatest need for viral load tests, accounting for over 65% of the global HIV population, yet this gold standard test is expensive, labor-intensive, and requires a laboratory to perform. Without access to these important tests, the effectiveness of ART can be compromised for patients in LMICs, potentially leading to an increase in HIV-related death.

RT-qPCR technology has been adapted for decentralized point-of-care (POC) tests to accommodate the growing need for viral load assays in LMICs. While POC tests reduce assay time and may simplify the workflow, they also require elaborate machinery that is more expensive than the traditional RT-qPCR tests. Viral load POC devices such as the GeneXpertR by Cepheid and the m-PIMA™ HIV-1/2 Viral Load Test by Abbott have eliminated much of the complicated workflow associated with PCR. However, the devices are expensive ($15K-$18K for equipment and $15-$25 per consumable), use many of the same temperature-sensitive reagents as the gold standard test, and require bulky equipment. This cost is incompatible with the requirements of the area of greatest need for these tests: Africa is home to approximately 87% of the global population that lives in extreme poverty, defined as a daily income of less than $1.90 a day.

Further challenges associated with the current state of the art in HIV testing include that existing assays are “singleplex” and not expandable beyond measurement of the viral load. An ideal assay platform would be modular and extensible, enabling simultaneous testing of HIV-associated comorbidities and standard serum markers of overall health. Here again, multiplex clinical assays, including the Luminex® system, require complex instrumentation, expensive consumables, and a complicated workflow.

One of the simplest assay formats suitable for POC implementation is the lateral flow assay. While highly successful, these assays are generally qualitative or at best semiquantitative, and difficult to multiplex. The introduction of methods for patterning fluidic channels in paper-based assays simplified the development of LFA-like multiplex tests and led to a rapid expansion of the field, but these assays still lack the sensitivity required for a useful HIV viral load test.

Thus, there exists a pressing need for novel devices and methods that enable POC HIV and other virus viral load measurements in a highly cost-effective, robust, and efficient format.

### SUMMARY

This disclosure addresses the above-mentioned need by providing devices and methods for viral nucleic acid isolation and detection. In one aspect, this disclosure provides a device for separating a nucleic acid of a virus from a fluid sample. In some embodiments, the device comprises (i) a first layer having a sample-receiving area adapted to receive a fluid sample and retain cells in the fluid sample; (ii) a second layer stacked below the first layer and having a viral-lysis area positioned at least in part in an overlapping manner with the sample-receiving area, wherein the viral-lysis area comprises a viral-lysis reagent; (iii) a third layer stacked below the second layer and having a pH-adjusting area in fluid connection with the viral-lysis area, wherein the pH-adjusting area comprises a pH-adjusting reagent; and (iv) a fourth layer stacked below the third layer and having a nucleic acid-receiving area in fluid connection with the pH-adjusting area. In some embodiments, the device further comprises a fifth layer having a wicking pad.

In some embodiments, the sample-receiving area of the first layer comprises (a) an upper filter pad adapted to receive the fluid sample and retain a first population of cells and (b) a lower filter pad stacked below the upper filter pad and adapted to retain a second population of cells.

In some embodiments, the viral-lysis area of the second layer comprises a lysis-reagent pad containing the lysing reagent. In some embodiments, the viral-lysis area further comprises an incubation pad stacked below the lysis-reagent pad.

In some embodiments, the lysis-reagent pad or the incubation pad comprises an incubation channel. In some embodiments, the incubation channel comprises a starting end positioned at least in part in an overlapping manner with the lysis-reagent pad and a terminating end extending laterally at a predetermined wicking distance from the starting end. In some embodiments, the terminating end is in fluid connection with the pH-adjusting area.

In some embodiments, the viral-lysis reagent comprises Triton X-100 (e.g., dried Triton X-100). In some embodiments, the lysis-reagent pad comprises fiberglass.

In some embodiments, the viral-lysis area is separated from the third layer by a degradable film.

In some embodiments, the pH-adjusting area of the third layer comprises a pH channel holding the pH-adjusting reagent. In some embodiments, the pH channel comprises an inlet end in fluid connection to the viral-lysis area in the third layer and an outlet end in fluid connection to the nucleic acid-receiving area in the fourth layer. In some embodiments, the pH-adjusting reagent comprises sodium acetate.

In some embodiments, the nucleic acid-receiving area of the fourth layer is functionalized.

In some embodiments, the fluid sample comprises a blood sample, a sputum sample, a urine sample, a urinary swab eluate sample, a nasal swab eluate sample, or a saliva sample. In some embodiments, the nucleic acid comprises RNA or DNA. In some embodiments, the virus comprises any one of HIV, Dengue, SARS-COV-2, and Ebola.

In some embodiments, the fifth layer comprises cellulose.

In some embodiments, the device further comprises a control member adapted to control passage of the fluid sample from the first layer into the second layer. In some embodiments, the control member is configured to receive a first user action to release the passage of the fluid sample from the first layer into the second layer. In some embodiments, the first user action activates a first timer and allows the fluid sample to contact with the viral-lysis reagent for a first predetermined period.

In some embodiments, the control member is configured to receive a second user action. In some embodiments, the second user action activates a second timer and allows the nucleic acid in the nucleic acid-receiving area to contact with a detecting agent for a second predetermined period.

In another aspect, this disclosure additionally provides a kit comprising the device described above and one or more probes having a sequence that is complementary to a target sequence of the nucleic acid.

In another aspect, this disclosure also provides a method for separating a nucleic acid of a virus from a fluid sample. In some embodiments, the method comprises (i) providing a fluid sample; (ii) contacting the fluid sample with the sample-receiving area in the first layer of the device described above; and (iii) incubating the fluid sample in the device under conditions permitting a fluid portion of the fluid sample to pass through the first layer, the second layer, and the third layer and to reach the fourth layer thereby obtaining the nucleic acid of the virus.

In yet another aspect, this disclosure further provides a method for detecting a nucleic acid of a virus in a fluid sample. In some embodiments, the method comprises: (a) providing the fourth layer obtained according to the method described above; (b) contacting the nucleic acid-receiving area with a reaction mixture; and (c) detecting a reaction between the nucleic acid and the reaction mixture.

In some embodiments, the reaction mixture comprises a probe having a sequence that is complementary to a target sequence of the nucleic acid. In some embodiments, the reaction mixture comprises a hybridization chain reaction (HCR) mixture.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1A, 1B, 1C, 1D, and 1E are a set of schematic views showing an example device for isolating and detecting viral nucleic acids.

FIG. 2 shows an example device workflow for isolating and detecting viral nucleic acids.

FIG. 3 is a schematic showing an in situ amplification via hybridization chain reaction (HCR) with a FRET induced fluorescent output.

FIG. 4 is a 3% agarose gel showing the product of HCR incubated with varying concentrations of synthetic HIV DNA and RNA sequences. In lanes 2, 4, and 6, a cocktail of 500 nM H1 and H2 was incubated for 1 hour with 100 nM, 50 nM, and 10 nM concentrations of synthetic DNA trigger, respectively. In lanes 3, 5, and 7, a cocktail of 500 nM H1 and H2 was incubated for 1 hour with 100 nM, 50 nM, and 10 nM concentrations of synthetic RNA trigger, respectively. Lane 8 contains the HCR reaction in the absence of any trigger sequence (incubated for 1 hour), and lane 1 contains a 100 base pair DNA ladder.

FIGS. 5A, 5B, 5C, and 5D are a set of diagrams showing roll-to-roll manufacturing of paper microfluidics.

FIGS. 6A, 6B, 6C, and 6D are a set of diagrams showing capture of oligonucleotides using Fusion 5 filters functionalized with chitosan. FIG. 6A shows staining with SybrSafe after running buffered DNA through the filters to empirically show 0.9, 3.5, and 6.1 μg DNA capture (brighter regions). FIG. 6B shows the absolute mass of DNA captured versus DNA mass filtered. The shaded region in FIG. 6B is the 95% confidence interval. FIG. 6C shows capture efficiency at different pH buffers using 50 μL of 1 μM yeast tRNA. FIG. 6D shows capture efficiency of DNA using 50 μL of 10 μM. All data points are the mean of an n=3 (except for “not functionalized” in FIG. 6D which is an n=2) with standard deviations represented with vertical lines, analyzed using JMP Pro 16.

FIG. 7 is a graph showing binned FRET intensity of HCR-FRET product on a Fusion 5 filter paper (Cytivia) using synthetic proviral DNA. Synthetic HIV proviral DNA was dried down on Fusion 5 filter paper. 5 μL of HCR solution was then applied to the filter paper as a sessile droplet and allowed to dry completely, and the filter paper was imaged with a fluorescence microscope. Raw fluorescence microscope images are shown on the right. Image intensities were quantified using ImageJ, and data is presented as the mean value with standard deviation represented by the vertical lines, analyzed using JMP Pro 16.

FIG. 8 is a graph showing binned FRET intensity of HCR-FRET product on Fusion 5 filter paper (Cytiva) using synthetic HIV RNA. Synthetic HIV RNA was dried down on Fusion 5 filter paper. 5 μL of HCR solution was then applied to the filter paper as a sessile droplet and allowed to dry completely, and the filter paper was imaged with a fluorescence microscope. Raw fluorescence microscope images are shown on the right. Image intensities were quantified using ImageJ, and data is presented as the mean value with standard deviation represented by the vertical lines, analyzed using JMP Pro 16.

FIGS. 9A, 9B, and 9C are a set of diagrams showing an example device and an example workflow for detecting nucleotides. FIG. 9A shows a device design for viral nucleic acid isolation and detection from whole blood with an integrated detection system. The pen is for scale. FIG. 9B shows internal porous media-based components of the device designed for passive nucleic acid isolation, including layers for plasma separation, viral lysis, and RNA capture. FIG. 9C shows an example device workflow (Steps 1-4).

### DETAILED DESCRIPTION

Existing methods for measuring viral load are complex, expensive, and not suitable for resource-limited environments or to the point of care (POC). To address the need for a POC-ready approach, this disclosure provides novel devices and methods for viral lysis and isolation as well as a simple, disposable, and nonenzymatic viral load assay based on a suitable reaction such as a hairpin cascade reaction or a toehold-triggered strand displacement reaction (TSDR).

### A. Devices for Isolating and Detecting Viral Nucleic Acids

Accordingly, in one aspect, this disclosure provides a device for separating a nucleic acid of a virus from a fluid sample. In some embodiments, the device comprises (i) a first layer having a sample-receiving area adapted to receive a fluid sample and retain cells or cell components in the fluid sample; (ii) a second layer stacked below the first layer and having a viral-lysis area positioned at least in part in an overlapping manner with the sample-receiving area, wherein the viral-lysis area comprises a viral-lysis reagent; (iii) a third layer stacked below the second layer and having a pH-adjusting area in fluid connection with the viral-lysis area, wherein the pH-adjusting area comprises a pH-adjusting reagent; and (iv) a fourth layer stacked below the third layer and having a nucleic acid-receiving area in fluid connection with the pH-adjusting area. In some embodiments, the device may additionally and/or optionally comprise a fifth layer having a wicking pad.

### First Layer

In some embodiments, the first layer of the device may be used for preprocessing a fluid sample, such as separating one or more components from a fluid sample. In one example, for a whole blood sample, the first layer may be used to separate plasma from white blood cells and/or red blood cells, or components thereof (e.g., cell debris). In some embodiments, separation of white blood cells and/or red blood cells, or components thereof, can be carried out using one or more filter pads.

In some embodiments, the filter pad may have an average pore size that functions as a filter and, for example, restricts flow of cells (e.g., white blood cells or red blood cells) or cell components thereof. In one example, the filter pad may have an average pore size that restricts the flow of white blood cells or cell components thereof. In some embodiments, the filter pad has an average pore size of about 8 μm to about 14 μm (e.g., 8 μm, 9 μm, 10 μm, 11 μm, 12 μm, 13 μm, 14 μm). In another example, the filter pad may have an average pore size that restricts the flow of red blood cells or cell components thereof. In some embodiments, the filter pad has an average pore size of about 0.2 μm to about 8 μm (e.g., 0.5 μm, 1 μm, 2 μm, 3 μm, 4 μm, 5 μm, 6 μm, 7 μm, 8 μm), or a single layer containing a gradient of decreasing pore size in any combination of the described pore sizes.

In some embodiments, the sample-receiving area of the first layer may include one or more filter pads to separate one or more components from a fluid sample. For example, the sample-receiving area of the first layer comprises an upper filter pad adapted to receive the fluid sample and retain a first population of cells and a lower filter pad stacked below the upper filter pad and adapted to retain a second population of cells. In some embodiments, the first population of cells may include white blood cells. In some embodiments, the second population of cells may include red blood cells. In some embodiments, to separate white blood cells and red blood cells, or components thereof, from a blood sample, the sample-receiving area of the first layer may include an upper filter pad having an average pore size (e.g., about 8 μm to about 14 μm) that restricts flow of white blood cells. The sample-receiving area of the first layer may further include a lower filter pad that is stacked below the upper filter pad and has an average pore size (e.g., about 0.2 μm to about 8 μm) that restricts flow of red blood cells.

In some embodiments, the sample-receiving area may have one or more openings with various geometric shapes and sizes. For example, the opening may have a circular, semi-circular, triangular, square, rectangular, pentagonal, or hexagonal shape. In some embodiments, the sample-receiving area has an opening with a circular shape. In some embodiments, the opening with a circular shape may have a diameter, e.g., from about 0.5 cm to about 10 cm (e.g., 1 cm, 2 cm, 3 cm, 4 cm, 5 cm, 6 cm, 7 cm, 8 cm, 9 cm, 10 cm).

In some embodiments, the opening of the sample-receiving area is configured to receive one or more filter pads. Accordingly, in some embodiments, the filter pad may have substantially the same size and shape as the opening of the sample receiving area. For example, the filter pad may have a circular, semi-circular, triangular, square, rectangular, pentagonal, or hexagonal shape. In some embodiments, the filter pad has a circular shape. In some embodiments, the filter pad with a circular shape may have a diameter, e.g., from about 0.5 cm to about 10 cm (e.g., 1 cm, 2 cm, 3 cm, 4 cm, 5 cm, 6 cm, 7 cm, 8 cm, 9 cm, 10 cm).

In some embodiments, the filter pad may have a size larger than the size of the opening of the sample receiving area. In some embodiments, one or more filter pads and the rest of the first layer can be formed as a unitary unit or as separate pieces.

The amount of fluid sample that is applied to the sample-receiving area may vary, so long as it is sufficient to provide for the desired capillary flow and operability of the assay. In some embodiments, the devices of the present disclosure are adapted for a small volume of sample, e.g., from 0.5 μL to 5 mL.

The sample may be applied to the sample application site using any convenient protocol, e.g., via a dropper, a pipette, a syringe, and the like. In addition, the fluid sample may be applied to the sample receiving area along with any suitable liquid, e.g., buffer, to provide for adequate fluid flow through the filter pad. Examples of any suitable liquid may include, without limitation, buffers, cell culture media (e.g., DMEM), etc. Examples of buffers include, but are not limited to tris, tricine, MOPS, HEPES, PIPES, MES, PBS, TBS, and the like. In one example, the suitable liquid may be mixed with the fluid sample before being applied to the sample-receiving area. In another example, the suitable liquid may be applied to the sample-receiving area concurrently with, before, or after applying the fluid sample.

The term “sample,” as used herein, includes a sample containing biological material. A sample may be, e.g., a fluid sample (e.g., a blood sample) or a tissue sample (e.g., a cheek swab). A sample may be a portion of a larger sample. In some embodiments, the fluid sample comprises a biological fluid, such as blood (e.g., whole blood), plasma, sputum, urine, sweat, urinary swab, semen, saliva, cheek swab, or combinations thereof. A sample can be a forensic sample or an environmental sample.

A sample can be preprocessed before it is introduced to the system. In some embodiments, the preprocessing can include extraction from a material that would not fit into the system, quantification of the amount of cells, DNA, RNA, or other biopolymers or molecules, concentration of a sample, separation of cell types, such as sperm from epithelial cells, or bead processing or other concentration methods or other manipulations of the sample.

A sample can be a biological sample having a nucleic acid, such as DNA or RNA. In some embodiments, the nucleic acid is RNA or DNA of a virus. Non-limiting examples of the virus include any one of Norwalk, Rotavirus, Poliovirus, Ebola virus, Marburg virus, Lassa virus, Hantavirus, Rabies, Influenza, Yellow fever virus, Coronavirus, SARS, SARS-COV-2, West Nile virus, Hepatitis A, C (HCV) and E virus, Dengue fever virus, toga (e.g., Rubella), Rhabdo (e.g., Rabies and VSV), Picorna (Polio and Rhinovirus), Myxo (e.g., influenza), retro (e.g., HIV, HTLV), bunya, corona and reoviruses which have profound effects on human health, including viroid like viruses such as hepatitis D virus and plant RNA viruses and viroids such as Tobus-, Luteo-, Tobamo-, Potex-, Tobra-, Como-, Nepo-, Almo-, Cucumo-, Bromo-, and Ilar-viruses.

In some embodiments, one or more components in the fluid sample to be removed may include cells. The term “cells,” as used in the context of biological samples, encompasses samples that are generally of similar sizes to individual cells, including but not limited to vesicles (such as liposomes), cells, virions, and substances bound to small particles such as beads, nanoparticles, or microspheres. In some embodiments, cells may include blood cells, cord blood cells, bone marrow cells, erythrocytes, white blood cells (leukocytes), lymphocytes, epithelial cells, stem cells, cancer cells, tumor cells, circulating tumor cells, progenitor cells, cells precursor, cord blood stem cells, hematopoietic stem cells, mesenchymal stem cells, adipose stem cells, pluripotent stem cells, induced pluripotent stem cells, embryonic stem cells, cells derived from umbilical cord, cells derived from fat tissues, matrix cells in stromal vascular fractions (SVF) cells in amniotic fluids, cells in menstrual blood, cells in cerebral spinal fluid, cells in urine, bone marrow stem cells, peripheral blood stem cells, CD34+ cells, colony forming cells, T cells, B cells, neural cells, immuno cells, dendritic cells, megakaryocytes, immobilized bone marrow cells, platelets, sperms, eggs, oocytes, microbes, microorganisms, bacteria, mold, yeast, protozoans, viruses, organelles, nuclei, nucleic acids, mitochondria, micelles, lipids, proteins, protein complexes, cell debris, parasites, fat roplets, multi-cellular organisms, spores, algae, clusters, aggregates of the above, industrial powders, polymers, powders, emulsions small droplets, dust, spherical particles (e.g., microspheres), fine particles, and colloidal (e.g., colloids).

### Second Layer

In some embodiments, the device of the present disclosure comprises a second layer stacked below the first layer and having a viral-lysis area positioned at least in part in an overlapping manner with the sample-receiving area.

In some embodiments, the viral-lysis area of the second layer comprises a lysis-reagent pad. In some embodiments, the lysis-reagent pad may include the lysing reagent.

In some embodiments, the lysing reagent may include a chemical or biochemical lysis reagent and a detergent/surfactant. Non-limiting examples of chemical lysis reagents that can be used for viron lysis and/or extraction of nucleic acid from virons include guanidinium salts (e.g., guanidinium thiocyanate and guanidinium hydrochloride) and urea. In some embodiments, the detergent/surfactant comprises a zwitterionic detergent/surfactant and/or a non-ionic detergent/surfactant. Examples of zwitterionic detergents/surfactants include without limitation 3-[(3-cholamidopropryl)dimethylammonio]-1-propanesulfonate (CHAPS), cocamidopropyl hydroxysultaine, and cocamidopropyl betaine. Non-limiting examples of non-ionic detergents/surfactants include polyoxyethylene glycol alkyl ethers (e.g., octaethylene glycol monododecyl ether and pentaethylene glycol monododecyl ether), polyoxypropylene glycol alkyl ethers, block copolymers of polyethylene glycol and polypropylene glycol (e.g., poloxamers (Pluronics)), polyoxyethylene glycol octylphenol ethers (e.g., Tritons, such as Triton X-100 and Triton X-114), polyoxyethylene glycol alkylphenol ethers (e.g., NP-40 and Nonoxynol-9), polyoxyethylene glycol sorbitan alkyl esters (e.g., Polysorbates/Tweens, such as Polysorbate/Tween 20), sorbitan alkyl esters (e.g., sorbitan monolaurate), glycerol alkyl esters (e.g., glyceryl laurate), and glucoside alkyl ethers (e.g., decyl glucoside, lauryl glucoside, and octyl glucoside). Non-limiting examples of ionic detergents include sodium dodecyl sulfate (SDS), lithium dodecyl sulfate (LDS), or deoxycholate (DOC). In some embodiments, the lysis reagent comprises a guanidinium salt (e.g., guanidinium thiocyanate or guanidinium hydrochloride), a Triton detergent/surfactant (e.g., Triton X-100 or Triton X-114), and CHAPS.

In some embodiments, the lysis reagent comprises a chemical or biochemical lysis reagent, a detergent/surfactant, and a buffering agent. In some embodiments, the buffering agent provides buffering in a basic pH range (e.g., about pH 8-11, about pH 8-10, about pH 10-11, about pH 9-10, or about pH 8-9). Non-limiting examples of buffering agents that provide buffering in a basic pH range include borate, N,N-bis(2-hydroxyethyl)glycine (bicine), N-tris(hydroxymethyl)methylglycine (tricine), tris(hydroxymethyl)methylamine (Tris), 3-amino-1-propanesulfonic acid, 4-(cyclohexylamino)-1-butanesulfonic acid (CABS), 3-(cyclohexylamino)-1-propanesulfonic acid (CAPS), 3-(cyclohexylamino)-2-hydroxy-1-propanesulfonic acid (CAPSO), 2-(cyclohexylamino)ethanesulfonic acid (CHES), N-(2-hydroxyethyl)piperazine-N′-(3-propanesulfonic acid) (EPPS), and 3-{[tris(hydroxymethyl)methyl]amino}-propanesulfonic acid (TAPS).

In some embodiments, the lysis reagent may include Triton X-100 (e.g., dried/lyophilized Triton X-100). In some embodiments, lysing efficiency may be improved by manipulating alkalinity of the lysis reagent. In some embodiments, the lysis reagent may include Triton X-100 and sodium hydroxide. In some embodiments, the lysis reagent may include SDS and/or Tween 20.

In some embodiments, the lysis reagent may further include a protease. The term “protease,” “peptidase,” or “proteinase,” as used herein, refers to an enzyme that catalyzes (increases the rate of) proteolysis, the breakdown of proteins into smaller polypeptides or single amino acids. The protease can be any one of serine proteases, cysteine proteases, threonine proteases, aspartic proteases, glutamic proteases, metalloproteases, asparagine peptide lyases, and combinations thereof.

In some embodiments, the lysing agent can be in an aqueous, dry, initially dry, solid, or gel form.

In some embodiments, the lysis-reagent pad can be formed of any suitable material. In some embodiments, the lysis-reagent pad comprises fiberglass, synthetic fiber, or a combination thereof.

In some embodiments, the lysis-reagent pad may include one or more incubation channels. In some embodiments, the incubation channel may include a starting end positioned at least in part in an overlapping manner with the lysis-reagent pad and a terminating end extending laterally at a predetermined wicking distance from the starting end, wherein the terminating end is in fluid connection with the pH-adjusting area.

In other embodiments, the viral-lysis area further includes an incubation pad stacked below the lysis-reagent pad. In some embodiments, the incubation pad comprises one or more incubation channels. In some embodiments, the incubation channel may include a starting end positioned at least in part in an overlapping manner with the lysis-reagent pad and a terminating end extending laterally at a predetermined wicking distance from the starting end, wherein the terminating end is in fluid connection with the pH-adjusting area.

The incubation channel provides a fluid path for the sample and lysis reagent mixture to wick from the lysis-reagent pad or the incubation pad, while allowing time for virions to be lysed. Accordingly, the dimension of the incubation channel, including wicking distance, channel shape, and channel width, may be determined based on the incubation time required for a lysing reagent to lyse at least a portion (e.g., at least 20%, 30%, 40%, 50%, 60%, 70%, 80%, 90%, 95%) of virions in a sample. In some embodiments, the dimension of the incubation channel is selected based on one or more characteristics of the material of the disclosed layers (e.g., porous materials, average pore size, pore size distribution, porosity, thickness, and wicking rate).

In some embodiments, the incubation channel may have various geometric shapes and sizes. For example, the incubation channel can be a serpentine-shaped channel, a straight or substantially straight channel, a Zig-Zag-shaped channel, or a combination thereof.

The term “wicking,” as used herein, refers to the movement of fluid through a porous medium as a result of capillary forces occurring in the pores of the medium. Typically, a porous medium has some degree of capillarity to the extent that fluid moves through the medium due to capillary forces created by, for example, small diameter pores or the close proximity of fibers. The term “wicking rate,” refers to the fluid movement per unit time, or, i.e., how far a fluid has traveled in a specified period of time.

In some embodiments, the incubation time needed for lysing virions can be achieved by including a degradable film under that viral-lysis area to separate it from the third layer. The degradable film can be degraded in a period of time after contacting a liquid in a fluid sample and subsequently releasing the fluid to the third layer. The term “film,” as used herein, includes thin films and sheets, in any shape, including rectangular, square, or other desired shape. The films described herein may be of any desired thickness and size. For example, the films may have a relatively thin thickness of about 0.1 μm to about 1 mm. Films may be in a single layer or multi-layered.

In some embodiments, the film may include a water-soluble polymer matrix. As used herein, the term “water-soluble” refers to substances that are at least partially dissolvable in a solvent, including but not limited to water. Examples of water-soluble polymers include, but are not limited to, polyethylene oxide, pullulan, hydroxypropylmethylcellulose, hydroxyethylcellulose, hydroxypropyl cellulose, polyvinyl pyrrolidone, carboxymethyl cellulose, polyvinyl alcohol, sodium alginate, polyethylene glycol, xanthan gum, tragacanth gum, guar gum, acacia gum, Arabic gum, polyacrylic acid, methylmethacrylate co-polymer, carboxyvinyl co-polymer, starch, gelatin, and combinations thereof. Examples of useful water-insoluble polymers include, but are not limited to, ethylcellulose, hydroxypropyl ethylcellulose, cellulose acetate phthalate, hydroxypropyl methylcellulose phthalate, and combinations thereof.

### Third Layer

In some embodiments, the device of the present disclosure comprises a third layer stacked below the second layer and having a pH-adjusting area in fluid connection with the viral-lysis area.

In some embodiments, the pH-adjusting area of the third layer may include a pH channel. The pH channel may include an inlet end in fluid connection to the viral-lysis area of the third layer and an outlet end in fluid connection to the nucleic acid-receiving area of the fourth layer.

In some embodiments, the pH channel may further include a pH-adjusting reagent. For example, the pH-adjusting reagent may be provided to increase the acidity of the fluid, e.g., to pH 5.0 to pH 6.5. Non-limiting examples of the pH-adjusting reagent may include acetic acid, boric acid, citric acid, lactic acid, phosphoric acid, hydrochloric acid, or salts derived therefrom. In some embodiments, the pH-adjusting reagent may include sodium acetate. In some embodiments, the pH-adjusting reagent can be in an aqueous, dry, initially dry, solid, or gel form.

In some embodiments, the pH channel may have various geometric shapes and sizes. For example, the pH channel can be a serpentine-shaped channel, a straight or substantially straight channel, a Zig-Zag-shaped channel, or a combination thereof.

### Fourth Layer

In some embodiments, the device of the present disclosure further comprises a fourth layer stacked below the third layer and having a nucleic acid-receiving area in fluid connection with the pH-adjusting area.

In some embodiments, the nucleic acid-receiving area may further include a nucleic acid adsorption pad capable of adsorbing viral nucleic acid molecules while allowing the bulk solution to pass through. The nucleic acid adsorption pad can be formed of any suitable materials. For example, the nucleic acid adsorption pad may be formed of paper, cloth, woven, or non-woven cellulose substrates. In some embodiments, the nucleic acid adsorption pad may be formed of paper, such as commercially available filter paper (e.g., Whatman paper, nitrocellulose, or fiberglass).

In some embodiments, the nucleic acid-receiving area, including the nucleic acid adsorption pad, may be functionalized so as to facilitate attachment of nucleic acid. For example, the nucleic acid-receiving area may be functionalized to be positively charged with, e.g., chitosan or polyethyleneimine (PEI).

The term “functionalized” or “chemically functionalized,” as used herein, means addition of functional groups onto the surface of a material by chemical reaction(s). As will be readily appreciated by a person skilled in the art, functionalization can be employed for surface modification of materials in order to achieve desired surface properties, such as biocompatibility, wettability, and so on.

In some embodiments, the fourth layer is removably stacked below the third layer so that the fourth layer can be detached as needed. For example, after the fourth layer is detached, one or more detection reagents may be added to the nucleic acid-receiving area to enable or facilitate detection (e.g., in situ detection) of viral nucleic acids. In some embodiments, the detection reagent may include an HCR mixture.

### Fifth Layer

In some embodiments, the device of the present disclosure may additionally and/or optionally comprise a fifth layer having a means (e.g., wicking pad, centrifugation, vacuum) to facilitate the bulk solution of the fluid sample to pass through the nucleic acid-receiving area of the fourth layer, e.g., ensuring that a plasma sample is pulled through the nucleic acid-receiving area of the fourth layer.

In some embodiments, the fifth layer may include a wicking pad. In some embodiments, the wicking pad can be formed of at least one absorbent material selected from paper, cotton, cellulose, cellulose nitrate, a cellulose fiber derivative, nylon (e.g., nylon wool), sintered glass, fiberglass, sintered polymer, sintered metal, and a synthetic polymer. In some embodiments, the fifth layer comprises cellulose.

In some embodiments, the above described first, second, third, fourth, and fifth layers may be formed of any suitable materials, such as paper, cotton, cellulose, cellulose nitrate, a cellulose fiber derivative, nylon (e.g., nylon wool), sintered glass, fiberglass, sintered polymer, sintered metal, and a synthetic polymer. In some embodiments, the above layers may also include a membrane (e.g., nitrocellulose, polyvinylidene fluoride, nylon, and polysulfone), glass, plastic (e.g., polyethylene terephthalate, polypropylene, polystyrene, and polycarbonate), silicon, metal, and metal oxide.

In some embodiments, the above described first, second, third, fourth, and fifth layers may be held together by any suitable fixation means. For example, the layers may be held together with adhesive (e.g., glue). In some embodiments, the device of the present disclosure may include one or more adhesive layers positioned between the first, second, third, fourth, and/or fifth layers. In some embodiments, two or more of the above described layers may be fabricated as a single piece.

In some embodiments, the device of the present disclosure may be housed in an enclosure. In some embodiments, the enclosure may be a cassette or cartridge. In some embodiments, the enclosure may include an opening for accessing the sample-receiving area of the first layer. In some embodiments, the enclosure allows the fourth layer to be detached from other layers so that the viral nucleic acids absorbed on the nucleic acid-receiving area can be subject to further analysis.

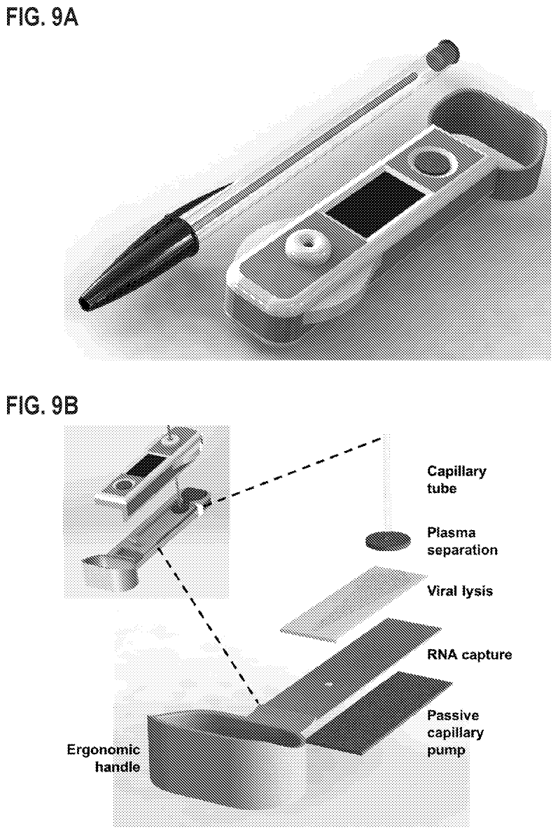

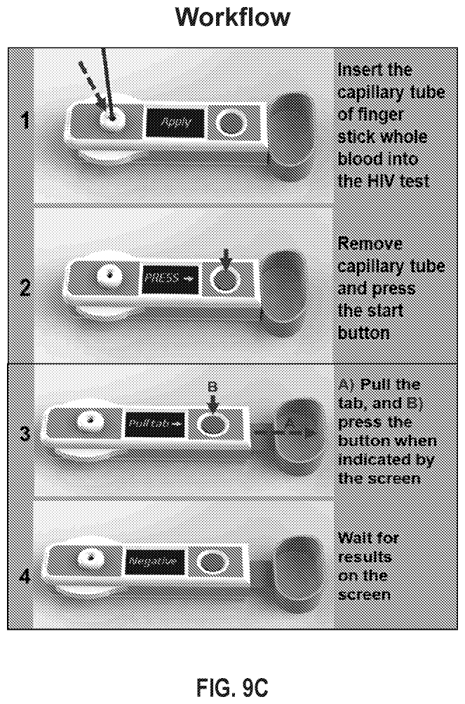

In some embodiments, the device further comprises a control member adapted to control passage of the fluid sample from the first layer into the second layer. For example, the control member can be a button, as shown FIG. 9C (“Start Button”). In some embodiments, the control member can be disposed on the enclosure for easy access by the user.

In some embodiments, the control member is configured to receive a first user action to release the passage of the fluid sample from the first layer into the second layer. In some embodiments, the first user action activates a first timer and allows the fluid sample to contact with the viral-lysis reagent for a first predetermined period. For example, the user may activate the control member (e.g., button) by a pressing action to start the flow of the fluid sample from the first layer to the second layer. The control member advantageously allows the user to control the start time of the detection process, such as when to start the lysis step. In addition, through the control member, the first user action can also activate the internal timer that controls the duration (the first predetermined period) of incubation of the fluid sample and the lysis reagent. The first predetermined period is a time period suitable for incubation of the fluid sample with the lysis reagent, such that a cell or virus can be adequately lysed, and nucleic acids can be released for capture and detection. In some embodiments, the first predetermined period can be 10, 20, 30, 40, 50, or 60 minutes.

In some embodiments, the control member is configured to receive a second user action. In some embodiments, the second user action activates a second timer and allows the nucleic acid in the nucleic acid-receiving area to contact with a detecting agent for a second predetermined period. For example, the second user action can be a pressing action that allows the user to start an internal timer that control the duration (the second predetermined period) of incubation of the captured nucleic acids with the detecting agent (e.g., HCR mixture). In some embodiments, the second predetermined period can be 30, 45, 60, or 75 minutes.

Also provided in this disclosure is a kit for isolating and/or detecting viral nucleic acids. In some embodiments, the kit may include the device as described above. In some embodiments, the kit may further include one or more detection reagents. In some embodiments, the detection reagent may include an HCR mixture. In some embodiments, the HCR mixture may include one or more probes for detecting viral nucleic acids. In some embodiments, the probe may have a sequence complementary to a target sequence of a viral nucleic acid.

In some embodiments, the kit also includes one or more additional agents contained in the same or different container from the detection agent. For example, the kit may include a detection reagent provided in a separate container or a separate compartment from the additional agents.

In some embodiments, the kit may optionally include an apparatus for collecting a sample (e.g., biological sample). In some embodiments, the apparatus for collecting a sample may include, without limitation, a capillary tube, a pipette, a syringe, a needle, a pump, and a swab. In some embodiments, the kit may include informational material. The informational material can be descriptive, instructional, marketing or other material that relates to the devices and/or methods of use thereof described herein.

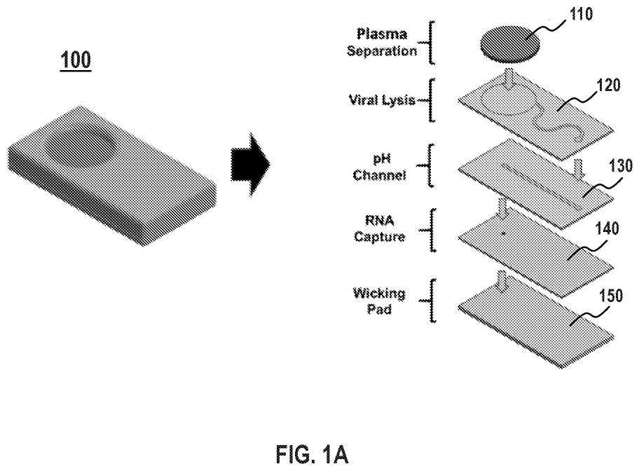

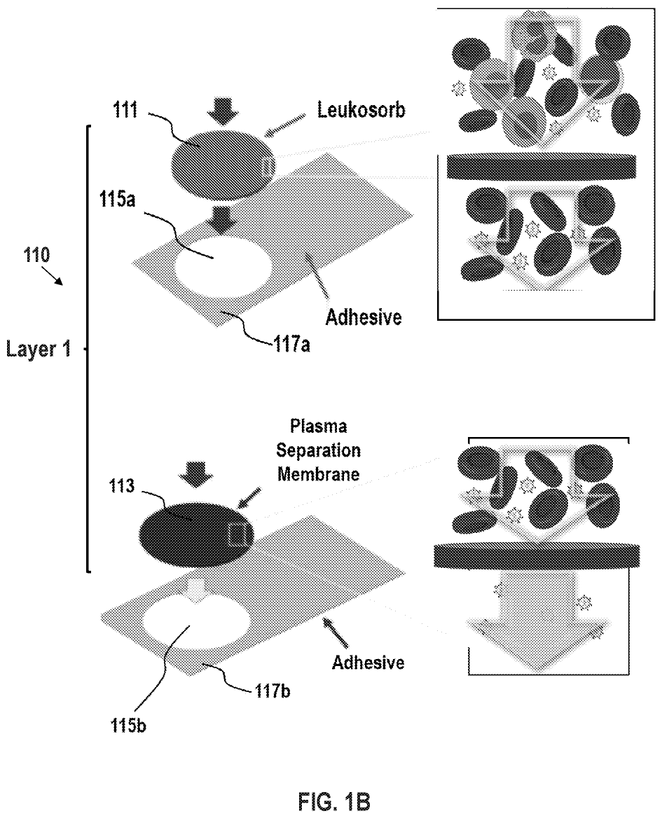

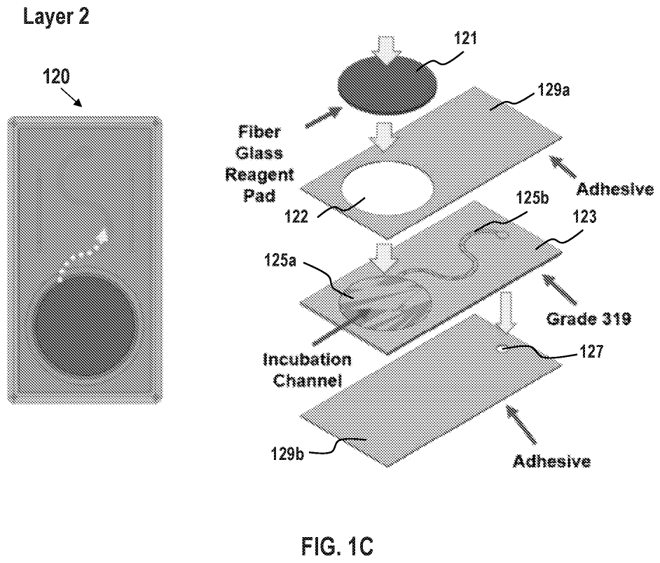

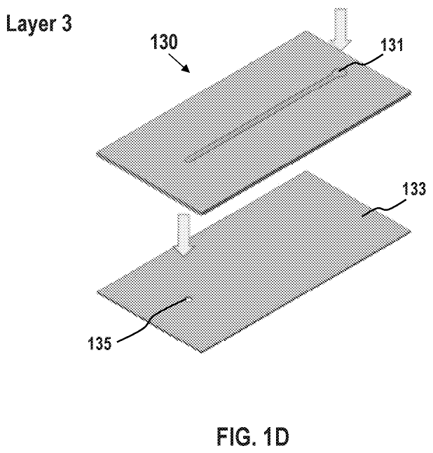

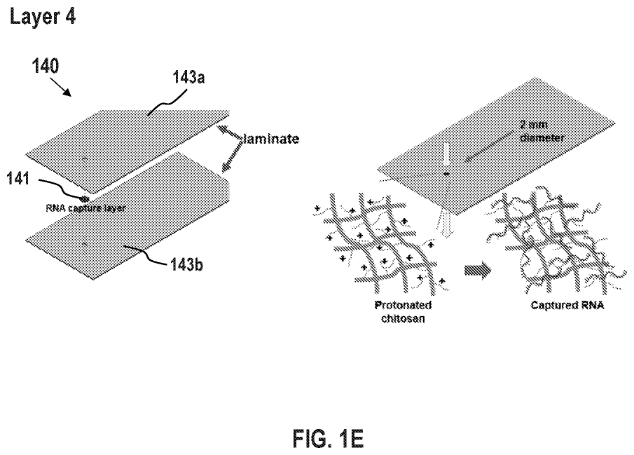

Referring now to FIG. 1A, device 100 comprises a first layer 110 (Layer 1) for sample preprocessing (e.g., plasma separation), a second layer 120 (Layer 2) for vial lysis, a third layer 130 (Layer 3) for pH adjustment, a fourth layer 140 (Layer 4) for nucleic acid capture. In some embodiments, the device 100 may further include a fifth layer 150 (Layer 5) which may include a wicking pad.

In some embodiments, the first layer 110 comprises a sample-receiving area adapted to receive a fluid sample and retain cells or cell components in the fluid sample. In some embodiments, the second layer 120 is stacked below the first layer 110 and having a viral-lysis area positioned at least in part in an overlapping manner with the sample-receiving area, wherein the viral-lysis area comprises a viral-lysis reagent. In some embodiments, the third layer 130 is stacked below the second layer 120 and having a pH-adjusting area in fluid connection with the viral-lysis area, wherein the pH-adjusting area comprises a pH-adjusting reagent. In some embodiments, the fourth layer 140 is stacked below the third layer 130 and having a nucleic acid-receiving area in fluid connection with the pH-adjusting area. In some embodiments, the additional or optional fifth layer 150 is stacked below the fourth layer 140 receiving the fluid passing the nucleic acid-receiving area of the fourth layer 140.

Referring now to FIG. 1B, in the scenarios where the fluid sample is a whole blood sample, the first layer 110 is adapted for plasma separation. As shown in FIG. 1B, the first layer 110 may include one or more filter pads (e.g., filter pads 111 and 113) having an average pore size suitable for retaining cells or cell components thereof (e.g., cell debris) from the fluid sample, e.g., by restricting the flow of cells or cell components thereof from passing through the filter pad.

For example, the filter pad 111 may be adapted to retain a first population of cells or cell components thereof, such as white blood cells. The filter pad 113 may be adapted to retain a second population of cells or cell components thereof, such as red blood cells.

In some embodiments, the filter pad may be harbored by an opening of an adhesive layer. For example, the filter pads 111 and 113 can be harbored, respectively, by the opening 115a of the adhesive layer 117a and the opening 115b of the adhesive layer 117b (FIG. 1B).

In some embodiments, the first layer 110 separates the plasma from the whole blood sample using two circular filter pads (i.e., filter pads 111 and 113) of about 1 cm to about 6 cm (e.g., 3 cm) in diameter, stacked one on top of the other. In one example, a sample of whole blood (e.g., about 250 μL to about 2000 μL) is applied to the uppermost pad (Leukosorb, Pall Corporation) (i.e., filter pad 111) where the red blood cells and plasma pass through to the next pad (i.e., filter pad 113) and the white blood cells are retained in the pad (i.e., filter pad 111). The second pad (i.e., filter pad 113) (CytoSep 1668 HV+, Ahlstrom-Munksjo) (i.e., filter pad 113) captures and retains red blood cells and allows plasma and its smaller components (e.g., virions) to pass through.

Referring now to FIG. 1C, the second layer 120, stacked below the first layer 110, may include a viral-lysis area positioned at least in part in an overlapping manner with the sample-receiving area of the first layer 110. In some embodiments, the viral-lysis area may include a lysis-reagent pad 121, which can be received by an opening 122 on an adhesive layer 129a.

In some embodiments, the lysis-reagent pad 121 may include the lysing reagent. In some embodiments, the lysis reagent may include Triton X-100 (e.g., dried/lyophilized Triton X-100). In some embodiments, the lysis reagent may include Triton X-100 and sodium hydroxide. In some embodiments, the lysing agent can be in an aqueous, dry, initially dry, solid, or gel form. In some embodiments, the lysis-reagent pad 121 may be formed of any suitable material. In some 20) embodiments, the lysis-reagent pad 121 comprises fiberglass, synthetic fiber, or a combination thereof.

In other embodiments, the viral-lysis area further includes an incubation pad 125a stacked below the lysis-reagent pad. In some embodiments, the incubation pad 125a comprises an incubation channel 125b. In some embodiments, the incubation channel may have various shapes, such as a serpentine, straight or substantially straight, or Zig-Zag shape or a combination thereof. In some embodiments, the incubation channel 125b is a serpentine-shaped channel.

The incubation pad 125a and the incubation channel 125b can be harbored on a substrate 123 of any suitable materials, such as paper (e.g., Ahlstrom-Munksjo Grade 319 chromatography paper), cotton, cellulose, cellulose nitrate, a cellulose fiber derivative, nylon (e.g., nylon wool), sintered glass, fiberglass, sintered polymer, sintered metal, and a synthetic polymer.

In some embodiments, the second layer 120 is stacked above the third layer 130 via an adhesive layer 129b, having an opening 127 that allows the liquid to pass through to the third layer 130.

With the white blood cells and red blood cells removed in the first layer 110, the plasma is left to travel via capillary action through the second layer 120. In some embodiments, the components of the second layer 120 may include a lysis-reagent pad 121 (e.g., formed of fiberglass) of about 1 mm to about 6 mm (e.g., 3 mm) and an incubation pad 125a (e.g., Grade 319, Ahlstrom-Munksjo). In one example, the lysis-reagent pad 121 may include dried lysing reagent (e.g., Triton X-100). The plasma passes through the lysis-reagent pad 121, rehydrating the lysing reagent and thus preparing the plasma for lysis. The plasma-lysis solution will wick through the incubation pad 125a in the incubation channel 125b (e.g., a serpentine channel) that allows time for the virions to be lysed. The time takes to lyse the virions (e.g., about 20 min to about 30 min), combined with a predictable wicking rate (e.g., about 10 mm to about 30 mm per minute), can be used to determine the dimensions and length of the incubation channel 125b of the incubation pad 125a. In some embodiments, the incubation channel 125b may be further patterned with hydrophobic barriers using a boronic acid polymer and applied using a roll-to-roll stamping method.

Referring now to FIG. 1D, the third layer 130 that is stacked below the second layer 120 may include a pH-adjusting area in fluid connection with the viral-lysis area. In some embodiments, the pH-adjusting area comprises a pH channel 131. In some embodiments, the pH channel 131 may further include a pH-adjusting reagent. For example, the pH-adjusting reagent may be provided to increase the acidity of the fluid. Non-limiting examples of the pH-adjusting reagent may include acetic acid, boric acid, citric acid, lactic acid, phosphoric acid, hydrochloric acid, or salts derived therefrom. In some embodiments, the pH-adjusting reagent may include sodium acetate. In some embodiments, the pH-adjusting reagent can be in an aqueous, dry, initially dry, solid, or gel form.

In some embodiments, the third layer 130 is adapted to prepare the lysed plasma sample for RNA capture by increasing the sample's acidity (e.g., pH 5.5 to 6.0). In one example, the third layer 130 can be formed of paper (e.g., Grade 319, Ahlstrom-Munksjo) having a hydrophilic channel containing dried reagent (e.g., sodium acetate) that will rehydrate and mix as the plasma travels through it.

In some embodiments, the third layer 130 may further include an adhesive layer 133 having an opening 135 to allow fluid to pass through to the fourth layer 140.

Referring now to FIG. 1E, the fourth layer 140 that is stacked below the third layer 130 includes a nucleic acid-receiving area in fluid connection with the pH-adjusting area of the third layer 130. In some embodiments, the nucleic acid-receiving area may further include a nucleic acid absorption pad 141 capable of absorbing viral nucleic acid molecules while allowing the bulk solution to pass through. The nucleic acid absorption pad 141 can be formed of paper, cloth, woven, or non-woven cellulose substrates. In some embodiments, the nucleic acid absorption pad 141 may be formed of paper, such as commercially available filter paper (e.g., Whatman paper). In some embodiments, the nucleic acid-receiving area, e.g., the nucleic acid absorption pad 141, may be functionalized. For example, the nucleic acid-receiving area may be functionalized with chitosan or polyethylene imine (PEI).

In some embodiments, the nucleic acid absorption pad 141 may be harbored between two laminated adhesive layers, i.e., 143a and 143b.

In some embodiments, the fourth layer 140 may include a nucleic acid absorption pad 141 which is in form of a small disc (e.g., about 2 mm to 5 mm in diameter) of commercially available filter paper (e.g., Whatman Fusion 5, Cytivia) that has been functionalized with chitosan. As the acidic plasma solution is wicked through the fourth layer 140, the chitosan becomes protonated. The negatively charged viral RNA, along with all other nucleic acids in the sample, becomes electrostatically adsorbed to the nucleic acid absorption pad 141 while the bulk solution passes through.

### B. Methods for Isolating and Detecting Viral Nucleic Acids

In another aspect, this disclosure also provides a method for separating a nucleic acid of a virus from a fluid sample. In some embodiments, the method comprises (i) providing a fluid sample; (ii) contacting the fluid sample with the sample-receiving area in the first layer of the device described above; and (iii) incubating the fluid sample in the device under conditions permitting a fluid portion of the fluid sample to pass through the first layer, the second layer, and the third layer and to reach the fourth layer thereby obtaining the nucleic acid of the virus.

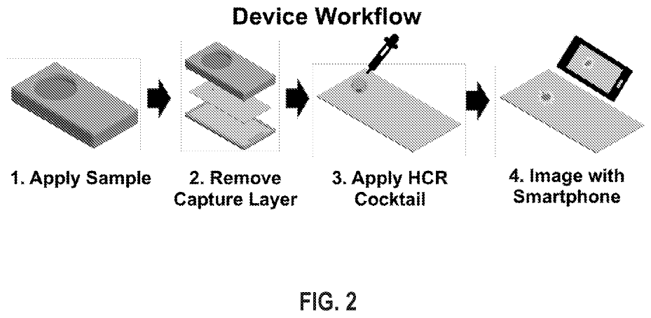

In yet another aspect, this disclosure further provides a method for detecting a nucleic acid of a virus in a fluid sample. In some embodiments, the method comprises: (a) providing the fourth layer obtained according to the method described above; (b) contacting the nucleic acid-receiving area with a reaction mixture; and (c) detecting a reaction between the nucleic acid and the reaction mixture (FIG. 2).

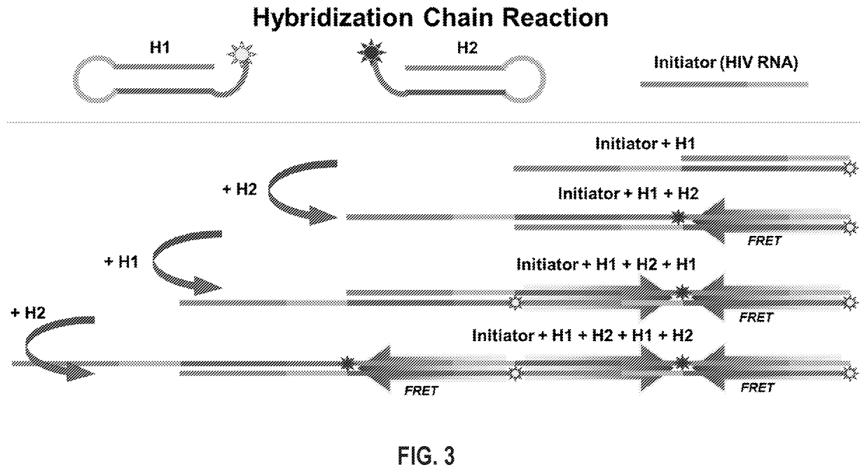

In some embodiments, detecting one or more viral nucleic acids in a biological sample is carried out in situ based on a nonenzymatic method of nucleic acid amplification termed a hybridizatrion chain reaction (HCR), as shown in FIG. 3. In this approach, a single copy of HIV RNA triggers opening of a probe DNA hairpin (H1) via duplex formation, which in turn triggers the opening of a second hairpin (H2). This then binds to a second molecule of H1, and the cascade continues. Each hairpin can be labeled at its termini with a fluorophore-quencher pair; thus, binding and opening of each hairpin, as the cascade progresses, causes fluorescence unquenching and an increase in signal for quantification by an imaging device, e.g., a smartphone camera, or by Förster Resonance Energy Transfer (FRET), whereby resonance energy is transferred from the electron of fluorophore A to fluorophore B and emitted as a photon with a longer wavelength than a photon emitted by fluorophore A.

In some embodiments, the reaction mixture comprises an HCR mixture. In some embodiments, the reaction mixture comprises one or more probes (e.g., probe set) having a sequence that is complementary to a target sequence of the nucleic acid. Exemplary sequences of HCR probes are provided in Table 1 below.

An “HCR probe set” or “HCR initiator/hairpin set” may include one or more initiator strands of nucleic acid together with one or more metastable HCR monomers, such as nucleic acid hairpins, that together are capable of forming the hybridization chain reaction polymer. HCR probes may be synthesized using standard methods, such as chemical nucleic acid synthesis, including commercial sources such as Integrated DNA Technologies (IDT, Coralville, Iowa), W.M. Keck Foundation Oligo Synthesis Resource (New Haven, Connecticut), or Molecular Instruments (Pasadena, California). Alternatively, the HCR probes may be synthesized and/or amplified using standard enzymatic methods, such as PCR followed by lambda exonuclease digestion of one strand to yield ssDNA, (see Current Protocols in Molecular Biology (2014): 14-23, hereby incorporated by reference in its entirety) or in vitro transcription followed by reverse transcription to yield ssDNA (see, e.g., Chen et al., Science 348:6233 (2015): aaa6090, hereby incorporated by reference in its entirety).

TABLE 1
Representative sequences of HCR probes

| SEQ | | |
|---|---|---|
| ID | | OTHER |
| NO | SEQUENCE | INFORMATION |
| 1 | HIV Trigger: 5′-AGGUUUGGGGAAGAGACA-3′ | HIV |
| 2 | H1: | (Target Gag- |
| | 5′-GGGGAAGAGACAAGGTTTTGTCTCTTCCCCAAACCT-3′ | gene) |
| 3 | H2: 5′- | |
| | AAACCTTGTCTCTTCCCCAGGTTTGGGGAAGAGACA-3′ | |
| 4 | H1 with FRET molecule: | |
| | 5′-GGGGAAGAGACAAGGTTTTGTCTCTTCCCCAAACCT- | |
| | ATTO 550-3′ | |
| 5 | H2 with FRET molecule: 5′-ATTO647N- | |
| | AAACCTTGTCTCTTCCCCAGGTTTGGGGAAGAGACA-3′ | |
| 6 | Trigger: 5′-UGACAUCAAGGACCUGCC-3′ | SARS-CoV-2 |
| 7 | H1: | (Target M-gene) |
| | 5′-CAAGGACCTGCCTGACATGGCAGGTCCTTGATGTCA-3′ | |
| 8 | H2: | |
| | 5′-ATGTCAGGCAGGTCCTTGTGACATCAAGGACCTGCC-3′ | |
| 9 | H1 with FRET molecule: | |
| | 5′-CAAGGACCTGCCTGACATGGCAGGTCCTTGATGTCA- | |
| | ATTO 550-3′ | |
| 10 | H2 with FRET molecule: | |
| | 5′-ATTO647N- | |
| | ATGTCAGGCAGGTCCTTGTGACATCAAGGACCTGCC-3′ | |

The sample may be applied to the sample application site using any convenient protocol, e.g., via a dropper, pipette, syringe, and the like. In addition, the fluid sample may be applied to the sample receiving area along with any suitable liquid, e.g., buffer, to provide for adequate fluid flow through the filter pad. Examples of any suitable liquid may include, without limitation, buffers, cell culture media (e.g., DMEM), etc. Examples of buffers include, but are not limited to tris, tricine, MOPS, HEPES, PIPES, MES, PBS, TBS, and the like. In one example, the suitable liquid may be mixed with the fluid sample before being applied to the sample-receiving area. In another example, the suitable liquid may be applied to the sample-receiving area concurrently with, before, or after applying the fluid sample.

Also provided in this disclosure is a method for identifying a patient having a viral infection or suspected of having a viral infection. In some embodiments, the method may include: (i) obtaining a sample (e.g., blood sample) for the patient; (ii) determining a level of the viral load (e.g., a level of HIV RNA) in the blood sample by a method described above; (iii) comparing the determined level of the viral load with a control level and determining whether the determined level is elevated as compared to the control level; and (iv) determining that the patient a viral infection if the determined level of the viral load is elevated as compared to the control level.

The terms “patient,” “individual,” and “subject,” are used interchangeably and generally refer to any living organism to which the disclosed methodology is utilized to obtain a bodily fluid sample in order to perform a diagnostic or monitoring method described herein. A patient can be an animal, such as a human. A patient may also be a domesticated animal or a farm animal. A “patient” or “individual” may also be referred to as a subject.

As used herein, a “control” level of a viral load refers, in some embodiments, to a level of viral load obtained from a sample obtained from one or more individuals who do not suffer from a disease or disorder that is of interest in the investigation, e.g., viral infection such as HIV infection. The level may be measured on an individual-by-individual basis or on an aggregate basis such as an average. A “control” level can also be determined by analysis of a population of individuals who have the disease or disorder but are not experiencing an acute phase of the disease or disorder. A “control” sample may be used to obtain such a “control” level. A “control” sample may be obtained from one or more individuals who do not suffer from a disease or disorder that is of interest in the investigation. A “control” sample can also be obtained from a population of individuals who have the disease or disorder but are not experiencing an acute phase of the disease or disorder. In some embodiments, a “control” level is from the same individual for whom a diagnosis is sought or whose condition is being monitored, but is obtained at a different time. In certain embodiments, a “control” level or sample can refer to a level or sample obtained from the same patient at an earlier time, e.g., weeks, months, or years earlier.

As used herein, “the determined level is elevated as compared to the control level” refers to a positive change in value from the control level.

### C. Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

As used herein, the term “contacting,” when used in reference to any set of components, includes any process whereby the components to be contacted are mixed into the same mixture (for example, are added into the same compartment or solution), and does not necessarily require actual physical contact between the recited components. The recited components can be contacted in any order or any combination (or sub-combination) and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the present disclosure.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of this disclosure unless otherwise claimed. No language in the specification should be construed as indicating any non-claimed element as essential to the practice of the invention. When used in this document, the term “exemplary” is intended to mean “by way of example” and is not intended to indicate that a particular exemplary item is preferred or required.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present disclosure. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### D. EXAMPLES

### Example 1

This example describes the materials and methods used in the subsequent examples below.

### Materials

Ultrapure Agarose and Sybr Safe DNA stain was purchased from Thermo Fisher Scientific (Waltham, MA, USA). 100 BP DNA ladder was purchased from VWR Life Science (Radnor, PA, USA). All DNA and RNA oligos, including DNA hairpins conjugated with ATTO 550 and ATTO 647N were purchased from Integrated DNA Technologies (Coralville, IA, USA). 10× TBE and ChemiDoc XRS gel imager were purchased from Bio-Rad Laboratories (Hercules, CA, USA). Sodium citrate, sodium chloride, tween 20, Bromophenol blue-Xylenecyanol, sucrose, and diethylpyrocarbonate were purchased from Sigma-Aldrich (St. Louis, MO, USA). The Fluoromax-4 fluorescence spectrometer was purchased from Horiba (Kyoto, Kyoto, Japan). Agarose gel casts, combs, MightSlim SX250 Power Supply, and HE 33 mini horizontal submarine unit were all purchased from Hoefer, Inc. (San Francisco, CA, USA). An Amplitron II thermocycler by Col-Parmer (Vernon Hills, IL, USA) was used for annealing. Fluorescence microscopy was done using an Olympus BX60 (Shinjuku, Japan) and a Spot Flex camera by Spot Imaging (Sterling Heights, MI, USA). All 3D printed materials were designed in SolidWorks 2018-2019 Student Edition and printed using polylactic acid (PLA) and a Prusa i3 MK3S purchased from Prusa (Prague, Czech Republic).

### Agarose Gel Electrophoresis

To create the 3% agarose gel, 1.2 grams of agarose was added to 40 mL of 1X TBE buffer and microwaved in 30 second increments until the agarose had been thoroughly dissolved and mixed in a molten agarose solution. 4 μL of SybrSafe was added to the molten agarose, mixed gently, and then poured into the gel cast and allowed to set for 1 hour. H1 and H2 hairpins were annealed by heating to 95° C. for 5 minutes and cooling to room temperature over 30 minutes. Each hairpin was then diluted to 1.5 μM concentrations in 5×SSCT. Synthetic DNA and RNA triggers were diluted to 300 nM, 150 nM, and 30 nM concentrations. Hairpins and triggers were added to cuvettes in 1:1:1 ratios for final trigger concentrations of 100 nM, 50 nM, and 10 nM DNA or RNA and final hairpin concentrations of 500 nM, as well as a 0 nM trigger control containing only the 500 nM hairpin concentrations and an equal ratio of 5×SSCT. The mixtures were mixed and incubated for 1 hour at room temperature, then 5 μL of each mixture were mixed with 10 μL of 1× loading buffer (100:1 ratio of 1% sucrose in Nanopure and Bromophenol blue-Xylenecyanol) and ran in the agarose gel at 150 V for 1 hour. The gel was then read with a ChemiDoc gel reader.

### HCR-FRET

Fluorophore conjugated H1 and H2 were mixed with synthetic RNA trigger in 5×SSCT to a final volume of 750 μL and final concentrations of 5 nM for each hairpin and 0, 1, 2, 3, 5, and 7 nM trigger concentrations and incubated in foil for 2 hours at room temperature in a 1.5 mL cuvette. The HCR reaction was added to a quartz cuvette and scanned using a Fluoromax-4 spectrofluorometer. The spectra were then analyzed using a custom script created using Matlab 2020b.

### Example 2

HIV viral load assays are composed of two primary components: 1) purification of the HIV target from whole blood, and 2) amplification of the analyte to quantifiable levels. Blood samples contain proteins and enzymes that will interfere with nucleic acid amplification assays if not removed prior to testing. To remove these contaminants, viral RNA isolation typically involves elaborate procedures designed to separate virions from whole blood, lyse the virions, and purify the viral RNA. After purifying the RNA, the amplification of viral signal is most commonly achieved using RT-qPCR. While effective, these methods involve a complex workflow, expensive reagents, and expensive and temperamental equipment to run.

To address the deficiencies of existing methods, this disclosure describes a hand-held diagnostic technology suitable for simple, enzyme-free detection of HIV viral load. However, this disclosure is not limited to viral load determination. The disclosed devices and methods are also suitable for other applications, such as determination of CD4 count, determination of HIV-associated comorbidities (for example, co-infection with tuberculosis) and protein markers of inflammation.

As shown in FIGS. 1A-E, the devices as disclosed herein may include an inexpensive, disposable assay strip. This assay strip leverages three novel developments for HIV viral load determination. The first of these is amplification of HIV genomic RNA using an HCR with fluorescence unquenching, an enzyme-free, DNA nanotechnology approach to nucleic acid detection. As shown in FIG. 3, the HCR relies on the reaction of an initiator sequence (the target HIV RNA) with two hairpins H1 and H2 to produce a DNA nanostructure. H1 and H2 are labeled at their termini with FRET donor-acceptor pairs; as the HCR process progresses, the hairpins unfold, producing an increasing acceptor fluorophore fluorescent signal. The initiator sequence binds to the toehold region of H1, causing H1 to unfold and thus revealing a sequence complementary to the toehold region of H2. As H2 binds, it unfolds, revealing a sequence complementary to the toehold region of H1. This allows the reaction to “cascade,” incorporating more copies of H1 and H2 into a fluorescent DNA nanostructure that is readily detectable. Hairpin DNA cascades are capable of significantly amplifying RNA signals, avoiding the need for enzymatic nucleic acid amplification and the sensitive reagents and equipment involved.

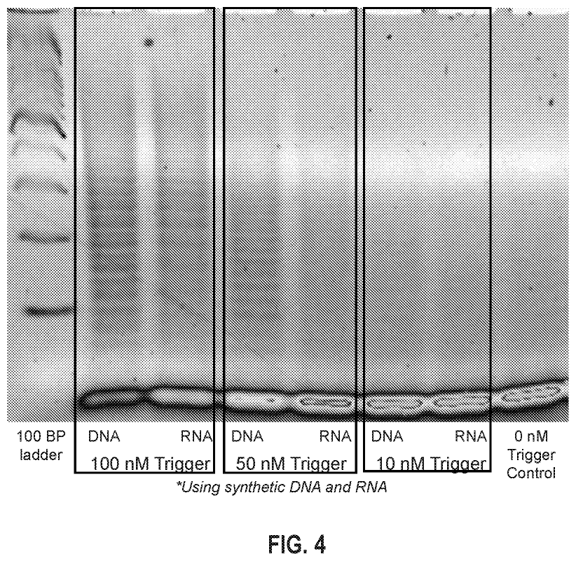

As demonstrated in this example, an HCR using the probe design methodology can successfully detect a synthetic nucleic acid sequence from HIV (FIG. 4). FIG. 4 shows the results of hairpin cascade reactions imaged in an agarose gel. DNA was visualized with SYBR Safe dye. In lanes 2, 4, and 6, a cocktail of 500 nM H1 and H2 was incubated for 1 hour with 100 nM, 50 nM, and 10 nM concentrations of synthetic DNA trigger, respectively. In lanes 3, 5, and 7, a cocktail of 500 nM H1 and H2 was incubated for 1 hour with 100 nM, 50 nM, and 10 nM concentrations of synthetic RNA trigger, respectively. Lane 8 contains the HCR reaction in the absence of any trigger sequence (incubated for 1 hour), and lane 1 contains a 100 base pair DNA ladder. Large molecular weight bands can be seen in the 100 nM and 50 nM DNA and RNA lanes, showing HCR products containing more than 27 hairpins. The HCR's ability to form hybridized chains in the presence of DNA and RNA triggers provide strong support for the feasibility of the approach.

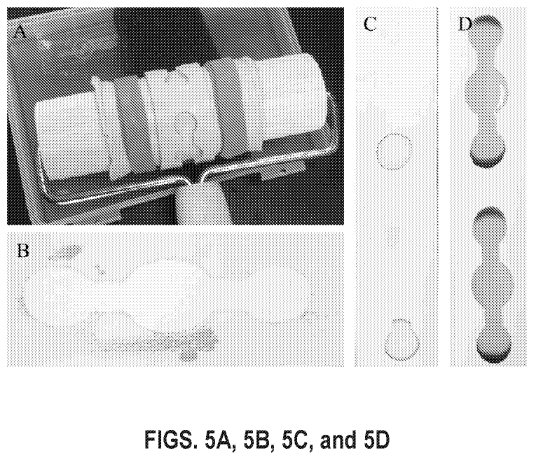

Additionally, the disclosed devices and methods may further employ patterning microfluidic channels in paper using a cellulose-binding boronic acid polymer, which is robust, compatible with roll-to-roll manufacturing and enables single-step production of assay consumables (FIGS. 5A-D). Imaging and analysis of the assay results can be performed using any suitable detection means, such as a standard smartphone camera with an integrated smartphone application.

### Example 3

To test capture of oligonucleotides with functionalized Fusion 5 filter paper, a Fusion 5 filter (Cytiva) was washed with 0.2 M NaOH on a rotary shaker for 5 minutes. The NaOH solution was aspirated, and the filter was washed with deionized water for 5 minutes on a rotary shaker three times. The filter was dried under vacuum at 50° C., and then incubated in a 0.25% w/v chitosan solution (Sigma-Aldrich) dissolved in 0.1 M acetic acid on a rotary shaker for 16 hours at room temperature. The filter was washed three times for 5 minutes using deionized water and dried under vacuum at 50° C. The resulting functionalized Fusion 5 filter was stored at 4° C. until used.

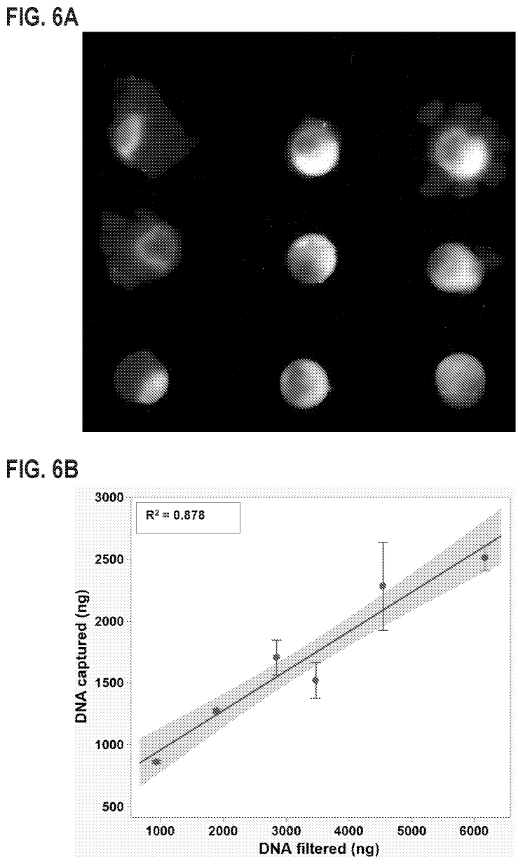

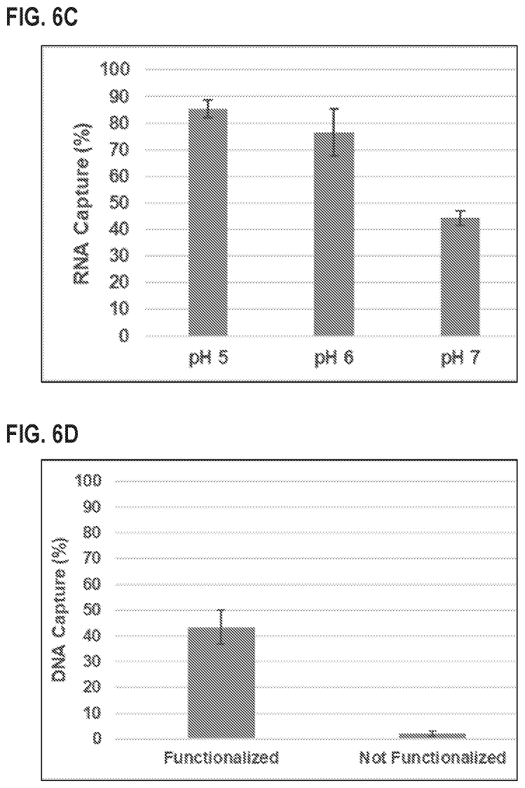

Filters from standard 0.22-micron centrifuge filters (Millipore Sigma) were removed and replaced with quarter-inch squares of the functionalized Fusion 5 filter. Stock DNA or RNA (30 nucleotide DNA and yeast tRNA) were diluted in 1× PBS (pH of 5, 6, or 7) and centrifuged through the filter at 1300×g. Nucleic acid concentrations were measured pre- and post-centrifugation using a Nanodrop Lite (Thermo Fisher Scientific). In FIG. 6A, filters were stained with 1× SybrSafe (Thermo Fisher Scientific) for 10 minutes and imaged using a ChemiDoc MP (Bio-Rad). Data from FIGS. 6B, 6C, and 6D were analyzed using JMP Pro 16.

The ability of the functionalized Fusion 5 filter paper to capture oligonucleotides is shown in FIGS. 6A-D. FIG. 6A empirically shows the captured nucleic acids on the used filters, and FIG. 6B shows the capture capability by mass. FIGS. 6C-D demonstrate the need for functionalization to electrostatically adsorb the short sequence nucleotides. FIG. 6C shows that the chitosan protonates at a pH below its pKa of ˜6.4. FIG. 6D shows that the short DNA and tRNA will pass through the filter unless it is functionalized with protonated chitosan.

### Example 4

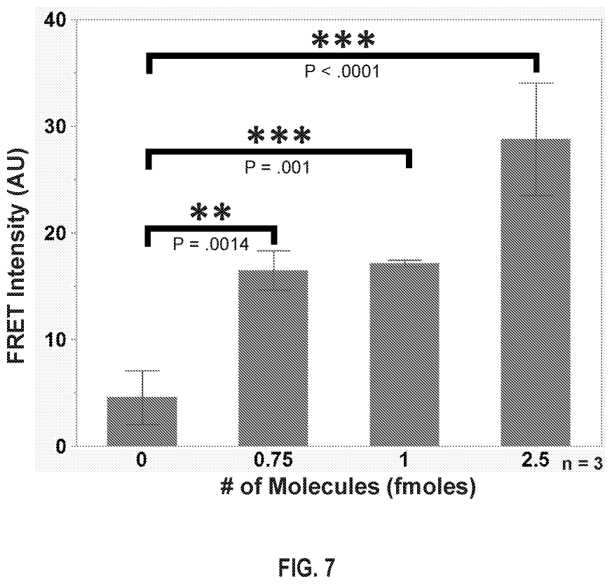

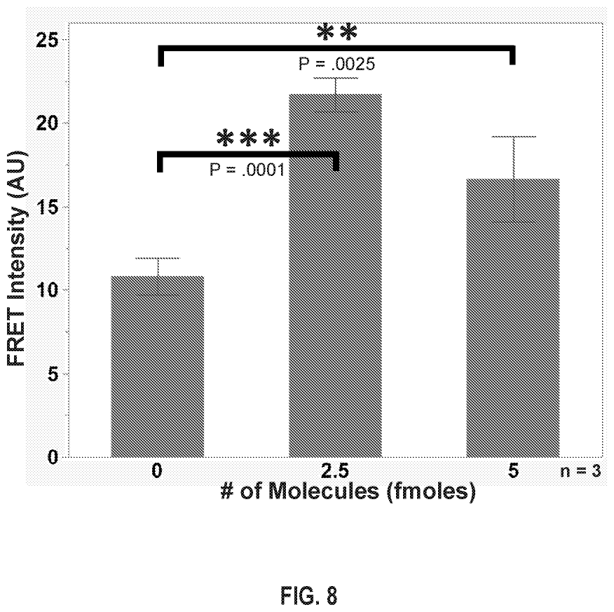

To test if the captured nucleic acids can be directly detected on the filter, 1.5 mm diameter pads were cut from Fusion 5 filter paper and adhered to 1.5 mm PDMS (Sigma-Aldrich) pedestals to allow for sessile droplet formation on the hydrophilic material. Synthetic trigger DNA or RNA was diluted using DI water and dried onto the paper pads. A 5 nM HCR solution of H1 and H2 hairpins was made in a DNA hybridization buffer (75 mM sodium chloride, 15 mM sodium citrate, 10 mM magnesium chloride, and 0.1% Tween 20 (all from Sigma-Aldrich)) or a RNA hybridization buffer (37.5 mM sodium chloride, 15 mM sodium citrate, 10 mM magnesium chloride, and 0.1% Tween 20) and 5 μL applied to each pedestal as a sessile droplet and allowed to dry completely in an enclosed environment surrounded by desiccant (Thermo Fisher Scientific). The dried paper was then imaged using a fluorescence microscope with a FRET cube and analyzed using ImageJ and JMP Pro 16. All p values were calculated using a two-tailed Student's t-test (n=3). All DNA and RNA were obtained from IDT. The Fusion 5 filters in FIGS. 7 and 8 were not functionalized with chitosan

H1:
(SEQ ID NO: 11)
5′-GGGGAAGAGACAAGGTTTTGTCTCTTCCCCAAACCT-3′-Cy3,
H2:
(SEQ ID NO: 12)
Cy5-5′-AAACCTTGTCTCTTCCCCAGGTTTGGGGAAGAGACA-3′,

DNA and RNA trigger:

(SEQ ID NO: 13)
AGGTTTGGGGAAGAGACA

and

(SEQ ID NO: 14)
AGGUUUGGGGAAGAGACA

As shown in FIGS. 7 and 8, both DNA and RNA trigger sequences can be detected directly on a Fusion 5 filter paper in the femtomole regime using the enzyme-free hybridization chain reaction incubated in a sessile droplet format and imaged using a fluorescence microscope.

### Example 5

This example demonstrates an example device (FIGS. 9A-B) and an example workflow (FIG. 9C) for detecting nucleotides. At Step 1 (FIG. 9C), to load a blood sample, a capillary tube of whole blood (for example, 150 μL) was inserted into the device. The blood was pulled from the capillary tube and into the device via capillary action, and then the button was pressed as described in FIG. 9C (Step 2) activating an internal timer within the device. In the first layer, the plasma was separated from the whole blood and pulled into the lysis layer, again via capillary action. When the internal timer ran out, a blister pack containing wash buffer was automatically punctured, releasing its contents into the lysis layer of the device, and flushing the lysed sample through the RNA capture layer. All liquid was wicked through the device by the capillary pump layer. The RNA capture layer was then removed from the other layers and into the detection zone of the device by the end user pulling the pull tab (FIG. 9C; Step 3). When the tab was pulled, the HCR solution was simultaneously released onto the RNA capture pad in the form of a sessile droplet. The end user then pressed the button again (FIG. 9C; Step 3), activating another internal timer. When the timer ran out, the device imaged the detection zone using an LED laser and photodiode and displayed the results of the assay on the LCD screen (FIG. 9C; Step 4).

The advantage of the disclosed nucleic acid test design is that it performs a complicated sample-to-answer nucleic acid test in an easy-to-use format that requires no training. The design uses inexpensive components, all capable of scaled manufacturing. The electronic components are inexpensive and similar to those used in a standard digital pregnancy test.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated herein in their entireties.

1. A device for separating a nucleic acid of a virus from a fluid sample, comprising
a first layer having a sample-receiving area adapted to receive a fluid sample and retain cells in the fluid sample;
a second layer stacked below the first layer and having a viral-lysis area positioned at least in part in an overlapping manner with the sample-receiving area, the viral-lysis area comprising a viral-lysis reagent;
a third layer stacked below the second layer and having a pH-adjusting area in fluid connection with the viral-lysis area, the pH-adjusting area comprising a pH-adjusting reagent; and
a fourth layer stacked below the third layer and having a nucleic acid-receiving area in fluid connection with the pH-adjusting area.

2. The device of claim 1, further comprising a fifth layer having a wicking pad.

3. The device of claim 1, wherein the sample-receiving area comprises (i) an upper filter pad adapted to receive the fluid sample and retain a first population of cells and (ii) a lower filter pad stacked below the upper filter pad and adapted to retain a second population of cells.

4. The device of claim 1, wherein the viral-lysis area comprises (iii) a lysis-reagent pad containing the lysing reagent.

5. The device of claim 4, wherein the viral-lysis area further comprises (iv) an incubation pad stacked below the lysis-reagent pad.

6. The device of claim 5, wherein the lysis-reagent pad or the incubation pad comprises an incubation channel having
a starting end positioned at least in part in an overlapping manner with the lysis-reagent pad, and
a terminating end extending laterally at a predetermined wicking distance from the starting end, wherein the terminating end is in fluid connection with the pH-adjusting area.

7. The device of claim 1, wherein the viral-lysis area is separated from the third layer by a degradable film.

8. The device of claim 1, wherein the pH-adjusting area comprises a pH channel holding the pH-adjusting reagent and having an inlet end in fluid connection to the viral-lysis area in the third layer and an outlet end in fluid connection to the nucleic acid-receiving area in the fourth layer.

9. The device of claim 1, wherein the nucleic acid-receiving area is functionalized.

10. The device of claim 1, wherein the fluid sample comprises a blood sample, a sputum sample, a urine sample, a urinary swab sample, or a saliva sample.

11. The device of claim 1, wherein the nucleic acid comprises RNA or DNA.

12. The device of claim 1, wherein the virus is HIV, Dengue, SARS-COV-2, or Ebola.

13. The device of claim 1, wherein the viral-lysis reagent comprises dried Triton X-100.

14. The device of claim 1, wherein the pH-adjusting reagent comprises sodium acetate.

15. The device of claim 2, wherein the fifth layer comprises cellulose.

16. The device of claim 4, wherein the lysis-reagent pad comprises fiberglass.

17. The device of claim 1, further comprising a control member adapted to control passage of the fluid sample from the first layer into the second layer.

18. The device of claim 17, wherein the control member is configured to receive a first user action to release the passage of the fluid sample from the first layer into the second layer.

19. The device of claim 18, wherein the first user action activates a first timer and allows the fluid sample to contact with the viral-lysis reagent for a first predetermined period.

20. The device of claim 17, wherein the control member is configured to receive a second user action and wherein the second user action activates a second timer and allows the nucleic acid in the nucleic acid-receiving area to contact with a detecting agent for a second predetermined period.

21. A kit comprising the device of claim 1 and a probe having a sequence that is complementary to a target sequence of the nucleic acid.

22. A method for separating a nucleic acid of a virus from a fluid sample, comprising,
providing a fluid sample,
contacting the fluid sample with the sample-receiving area in the first layer of the device of claim 1; and
incubating the fluid sample in the device under conditions permitting a fluid portion of the fluid sample to pass through the first layer, the second layer, and the third layer and to reach the fourth layer, thereby obtaining the nucleic acid of the virus.

23. A method for detecting a nucleic acid of a virus in a fluid sample, comprising
providing the fourth layer obtained according to the method of claim 22;
contacting the nucleic acid-receiving area with a reaction mixture, and
detecting a reaction between the nucleic acid and the reaction mixture.

24. The method of claim 23, wherein the reaction mixture comprises a probe having a sequence that is complementary to a target sequence of the nucleic acid.

25. The method of claim 24, wherein the reaction mixture is a hybridization chain reaction (HCR) mixture.


SEQUENCE LISTING (14 sequences)

SEQ ID NO: 1 - RNA, 18 nt, Artificial
  Synthetic
agguuuggggaagagaca

SEQ ID NO: 2 - DNA, 36 bp, Artificial
  Synthetic
ggggaagagacaaggttttgtctcttccccaaacct

SEQ ID NO: 3 - DNA, 36 bp, Artificial
  Synthetic
aaaccttgtctcttccccaggtttggggaagagaca

SEQ ID NO: 4 - DNA, 36 bp, Artificial; identical to SEQ ID NO: 2
  Synthetic

SEQ ID NO: 5 - DNA, 36 bp, Artificial; identical to SEQ ID NO: 3
  Synthetic

SEQ ID NO: 6 - RNA, 18 nt, Artificial
  Synthetic
ugacaucaaggaccugcc

SEQ ID NO: 7 - DNA, 36 bp, Artificial
  Synthetic
caaggacctgcctgacatggcaggtccttgatgtca

SEQ ID NO: 8 - DNA, 36 bp, Artificial
  Synthetic
atgtcaggcaggtccttgtgacatcaaggacctgcc

SEQ ID NO: 9 - DNA, 36 bp, Artificial; identical to SEQ ID NO: 7
  Synthetic

SEQ ID NO: 10 - DNA, 36 bp, Artificial; identical to SEQ ID NO: 8
  Synthetic

SEQ ID NO: 11 - DNA, 36 bp, Artificial; identical to SEQ ID NO: 2
  Synthetic

SEQ ID NO: 12 - DNA, 36 bp, Artificial; identical to SEQ ID NO: 3
  Synthetic

SEQ ID NO: 13 - DNA, 18 bp, Artificial
  Synthetic
aggtttggggaagagaca

SEQ ID NO: 14 - RNA, 18 nt, Artificial
  Synthetic
agguuuggggaagagaca